(19) United States
(12) Patent Application Publication
(10) Pub. No.: US 2021/0015947 A1
(43) Pub. Date: Jan. 21, 2021

(54) CONTRAST MEDIUM FOR MICROANGIOGRAPHY

(71) Applicants: FUMEDICA INTERTRADE AG, Cham (CH); UNIVERSITÄT BERN, Bern (CH)

(72) Inventors: Beat STEGER, Sobrio (CH); Ruslan HLUSHCHUK, Bern (CH); Valentin DJONOV, Bern (CH)

(73) Assignee: PITENGO GMBH, Beromünster (CH)

(21) Appl. No.: 16/491,866

(22) PCT Filed: Mar. 6, 2018
(86) PCT No.: PCT/EP2018/055463

§ 371 Date: Sep. 6, 2020

(30) Foreign Application Priority Data
Mar. 9, 2017 (CH) 00284/17

(51) Int. Cl. A61K 49/04

(52) U.S. Cl. CPC A61K 49/0438; A61K 49/0452

(57) ABSTRACT

The invention relates to a contrast medium for ex vivo microangiography for digital imaging of the vascular system of a mouse or rat or other laboratory animals, and of individual animal and human organs, comprising an iodized, esterified oil, a polyurethane, and a hardener. The invention further relates to a method for producing the contrast medium; to a kit of parts, comprising the various containers having the components of the contrast medium according to the invention that are to be mixed; and to a preferred use of the contrast medium according to the invention for imaging by means of a micro-CT device.

### TECHNICAL FIELD

The present invention relates to a contrast agent for post-mortem micro angiography, i.e. for the digital imaging of a vascular system, especially of small animals, such as for example a mouse, a rat, or other laboratory animals, as well as of single animal- and human organs, by means of an x-ray based imaging method, especially of a nano- or micro-CT-device.

### PRIOR ART

It is an aim of micro angiography to visualize smallest vessels, i.e. capillaries of single organs or of whole bodies in a three-dimensional manner, and to depict them in an exact manner for the purpose of analysis. Especially for the pharmacological research, however, also in forensics, an analysis of the intact vessels is necessary. Up to date, essentially two technologies were used for the imaging of micro vessels: on the one hand, the so-called casting method, and secondly the radiologic contrast agent imaging, especially the Microfil®-method.

3D-imaging methods such as Micro Computed Tomogaphy (micro-CT) have increasingly caused a stir in the past years. The imaging of the vascular system requires a perfusion with a radiopaque contrast agent for the visualization by means of micro-CT. In vascular research, micro-CT has so far been used in combination with various contrast agents, in order to represent the vascular system of various organs, such as e.g. brain, heart, liver, kidney, lungs, as well as of the rear extremities, as well as of tumors. However, these studies were always limited in terms of resolution, or due to an incomplete filling and faulty perfusion (also due to the relatively high viscosity of the contrast agent used) (e.g. Perrien, D. S., 2016).

In the vascular corrosion casting method, casting material, e.g. an agent to be polymerized, based on polyurethane, is injected into the vessels (e.g. Meyer et al., 2007 & 2008; Krucker et al., 2006). Subsequently, after its polymerization, the surrounding tissue is chemically macerated and digested. The die casts of the vessels thus produced are subsequently evaluated by scanning electron microscopy (SEM) or radiologically in a three-dimensional manner. Furthermore, the possibility exists to create a three-dimensional vessel model. The serious main disadvantage of this method occurs by the maceration of the tissue, because it excludes the option of a (subsequent) histological examination and thus also not even allows any localization within the tissue. A further disadvantage of this method when using scanning electron microscopy is the limited representability of vessels, as one does achieve an image of vessels at the outer surface of the vessel cast, however, not in the inside of the cast.

The Microfil®-method is especially useful for the representation of vessels with a diameter of up to 100 μm. As this vessel diameter hardly allows any conclusions about the region of the capillaries, the application is varied by the user in different ways. That means that the user varies the composition of the substance of the contrast agent according to his needs by dilution or other modification, such that the contrast agent can enter into smaller vessels. Generally, such a capillary region of up to 10 μm can be reached and represented. The type of dilution or modification of the contrast agent composition however, does not occur according to any known standard, but individually. The application usually is carried out manually. According to published studies, the perfusion, probably as a result of a relatively high viscosity, nevertheless is insufficient (Perrien, D. S. 2016).

The exchange of oxygen and metabolites mainly takes place at the level of the capillary bed, i.e. at vessel diameters of about 4 to 10 μm. Preclinical studies are usually limited to the representation of micro vessels of a medium diameter (about 15 μm). In the meantime, research has the possibility of using micro-CT-scanners or micro-CT devices, respectively, which can represent capillaries up to a diameter of 3-4 μm, provided that these capillaries are also reached and filled with a suitable added contrast agent.

Accordingly, there is a great need for a contrast agent which can penetrate far enough into the smallest vessels and allows as well as possible an artefact-free visualization of the micro vasculature or a morphometric 3D-image analysis, respectively, by micro-CT (see also Zagorchev et al., 2010).

### SUMMARY OF THE INVENTION

Accordingly, the present invention solves the problem of providing an improved contrast agent, which can penetrate into capillaries with a diameter of up to 3-4 μm, and thus allows a representation of the vessels by micro-CT method. There also is a great need for an improved ex vivo method for angiography, which allows a reproducible representation of the vascular system. With the development of the contrast solution according to the invention, such an improved contrast agent is provided, which overcomes the disadvantages of the current prior art, as well as an improved method for angiography.

The problem described above is solved by the contrast agent according to claim 1, or by the kit-of-parts as described in claim 10, respectively. The provision of the improved contrast agent is carried out preferably by the method of production according to claim 14, and the ex vivo method for micro angiography according to claim 15 allows a reproducible and thus optimized application of the contrast agent according to the invention.

The contrast agent according to the invention, which allows the representation of capillaries with a diameter of up to 3-4 μm, is used ex vivo. The contrast agent according to the invention for ex vivo or post-mortem micro angiography, respectively, preferably serves the digital imaging and thus the examination of the vascular system of a mouse or a rat, or other laboratory animals and of human organs by means of a micro-CT-device. The contrast agent according to the invention contains an iodized, esterified oil, preferably an iodized, esterified linseed oil or an iodized, esterified poppy seed oil. In addition, the contrast agent according to the invention contains a polyurethane and a hardener, as well as a ketone as a solvent. The ketone preferably is selected from the following group: butanone (or 2-butanone or methylethylketone or C₄H₈O, respectively), acetone (or 2-propanone or dimethylketone, or C₃H₆O, respectively), or 3-pentanone (or diethylketone or C₅H₁₀O, respectively). Therein, especially 2-butanone or acetone are preferred as a solvent, wherein 2-butanone is most preferred. Alternatively, possibly methylene chloride can be used as a solvent. The mixture of the iodized, esterified oil with the ketone is termed “contrast solution” for the purpose of this application.

An especially preferred embodiment of the contrast agent furthermore contains a dye, wherein the dye preferably is a blue dye (e.g. BlueDye of VasQtec). In case a dye is added, it also forms part of the mixture termed “contrast solution” for the purpose of this application.

A preferred embodiment contains, as mentioned above, an iodized, esterified linseed oil as an iodized, esterified oil. The contrast agent according to the invention therefore is an iodine-containing and preferably also dye-containing, polymerized substance, which preferably is based on iodized, esterified linseed oil. Preferably, the iodized, esterified linseed oil is ethyl-9,12,15-triiodo-octadecatrienoate, or ethyl-linoleate, respectively.

The contrast agent according to the invention preferably comprises auto-fluorescent properties and results in a preferably blue coloring of the vessels in the initial phase of application, which facilitates the optical control of the injection.

Prior to the application or injection, respectively, into the body to be examined, or into the organ to be examined, respectively, the contrast solution is mixed according to a defined scheme with said polyurethane (PU-) resin and with a hardener.

The polyurethane to be used for the production of the contrast agent according to the invention is preferably a polyisocyanate-pre-polymer. It therefore preferably is an aliphatic isocyanate. Preferably, the isocyanate comprises an aromatic or preferably an aliphatic group. In order for the polyurethane to remain flexible, normally polyethers are used. It is especially advantageous if the polyurethane contains a polyester, or preferably a polyether, especially preferably a polyether composed of ethylenglycol- and/or propylenglycol-residues. Preferably, the polyurethane comprises a chain-extender, especially a diol or preferably a diamine-based chain extender, especially preferably diethylmethylbenzolediamine. A contrast agent especially suitable for micro angiography contains 4,4′-methylene-di(cyclohexyl-isocyanate) (HDMI), or 4,4′-dicyclohexylmethane-diisocyanate, respectively, as a polyurethane.

The hardener used in the contrast agent, which preferably is only added to the mixture of the iodized, esterified oil, butanone and PU shortly before the injection into the body, or selectively into an organ, respectively, preferably is a modified, aromatic diamine, especially preferably a diethylmethylbenzoldiamine, for example 2,6-diamino-3,5-diethyltoluene. A hardener which is especially advantageous for the use in the contrast agent according to the invention comprises a mixture of two isomers of diethymethylbenzoldiamine, most preferably an isomeric mixture of 2,6-diamino-3,5-diethyltoluol and 2,4-diamino-3,6-diethyltoluol at a ratio of 7:3.

Advantageously, the iodized, esterified oil is contained in the contrast agent with 20-60%, preferably with 22-45%, especially preferably with 24-30% (in each case percent by volume). The ketone, or preferably the 2-butanone, respectively, preferably is contained with 7-30%, preferably with 10-25%, especially preferably with 14-22% in the contrast agent (in each case percent by volume).

The polyurethane is preferably contained with 25-60%, preferably with 35-50%, especially preferably with 38-50% and most preferably with 43-47% in the contrast agent (in each case percent by volume).

The hardener is preferably contained with 4-10%, preferably with 5-9%, especially preferably with 6-8% in the contrast agent (in each case percent by volume).

The invention further concerns a kit-of-parts for micro angiography, comprising:
a first container, containing the above mentioned iodized, esterified oil, and said ketone, or preferably the 2-butanone, respectively; and
a second container, containing the above mentioned polyurethane; and
a third container, containing the above mentioned hardener.

The first container therein contains preferably a first mixture of 2-4 ml, preferably 2.5-2.8 ml of the iodized, esterified oil, preferably of the iodized, esterified linseed oil, and 2-3 ml, preferably 2.2-2.9 ml of the ketone, or of the 2-butanone, respectively. In other words, the first container contains the “contrast solution”. Therein, the first container preferably additionally contains the above mentioned dye, preferably a blue dye, which in case of its admixture also forms part of the “contrast solution”. The addition of a pinch of the dye, which corresponds to about 0.2 g of the dye, already is sufficient for the present application. The second container contains preferably 4-7 ml, especially preferably 4.5-5 ml of the polyurethane; and the third container contains preferably 0.5-1.5 ml, especially preferably 0.8-1.2 ml of the hardener. Preferably the volume ratio of the polyurethane to the hardener in the mixture to be injected lies in the range of 100:10 to 100:25, especially preferably in the region of 100:16 to 100:19.

Preferably, the kit-of-parts furthermore contains
a first syringe for the reception of the contents of the first container and the second container, preferably a syringe with a volume of 12 ml;
a second syringe for the reception of the contents of the third container, preferably a syringe with a volume of 1 ml;
a mixing container for mixing the contents of the first syringe and the second syringe;
a dispenser for the control of the first syringe and the second syringe, wherein the dispenser comprises a device for the reception of, in each case, a first end of the first and of the second syringe;
and preferably an adapter element for the reception of, in each case, a second end of the first and of the second syringe and for the reception of a first end of the mixing chamber.

The invention furthermore concerns a method for the production of the above mentioned contrast agent for micro angiography, for the digital imaging of a vascular system of a mouse or of a rat by means of a micro-CT-(μCT-) device, wherein the method of production comprises the following steps:
a) provision of a first mixture of iodized, esterified oil, preferably of iodized, esterified linseed oil, with a ketone, preferably with 2-butanone, in a first container;
b) provision of a polyurethane in a second container;
c) provision of a hardener in a third container;
d) blending and mixing of the contents of the first container with the contents of the second container to form a second mixture;
e) blending of the contents of the third container with the second mixture of step d) in a mixing element, immediately prior to the injection into the vascular system of the mouse or rat; wherein preferably a mixing ratio of 100:16 of the polyurethane to the hardener is used.

The invention further concerns a method for micro angiography for the digital imaging of a vascular system of an animal body, especially of a mouse or a rat, by means of a micro-CT-device, comprising the following steps:
provision of the above mentioned contrast agent, preferably according to the above mentioned method;
insertion of a cannula and flushing or exsanguination, respectively, of the animal body to be examined, preferably with a clear solution, especially with PBS (phosphate buffered saline solution), wherein preferably for a mouse a flushing amount of 20-100 ml, and alternatively for a rat a flushing amount of 20-200 ml is used;
injection of the contrast agent, preferably at a constant flow rate and preferably under as constant a pressure as possible, wherein the flow rate preferably is at the most 3 ml/min, especially preferably at the most 1.5 ml/min.

The application or injection, respectively, of the contrast agent can either be carried out manually by means of a dispenser, or alternatively, by means of an injection pump or by means of a perfusor, respectively, which has preferably been modified or adjusted according to individual needs, respectively, by means of which a specific volume per time unit can be maintained.

For a mouse, normally between 1-12 ml of the contrast agent are injected, and for a rat normally between 1-30 ml of the contrast agent, depending on the organ of interest to be examined.

The injection of the contrast agent into the mouse or rat preferably is carried out during a time frame of 1-6 min, wherein preferably for a mouse or a rat an injection rate of 0.5-12 ml/min, especially preferably of 1-3 ml/min is used, most preferably of at the most 1.5 ml/min.

After the injection of the contrast agent, preferably, it is waited until a curing of the contrast agent in the animal body takes place. Next, the respective organ or the respective part of the body is cut out and chemically fixated, and subsequently is scanned by means of a micro-CT-device.

The contrast agent according to the invention in the first line is suitable for experimental purposes, i.e. in pre-clinical research for the perfusion of bodies of small animals, especially of mouse and rat. However, it can for example also be used for the selective perfusion of single regions or organs of larger experimental animals, such as e.g. of rabbits, dogs, fish, sheep, minipigs, etc. These are for example used in orthopedic or dental research (dental replacements, bone replacements, etc.). For this purpose, the contrast agent is specifically injected into the artery, of which the “target region” shall be represented. In this manner, a selective injection of contrast agent into single organs or parts of the body is possible, such as e.g. into the lower jaw, etc. and the corresponding organs or parts of the body can then be represented. The contrast agent according to the invention is also suitable for the application in forensics, and therefore also for the forensic examination of human bodies.

The provision of the contrast agent according to the invention opens up new regions of application for ex vivo micro-CT technology in various areas of biomedical research. While in the PU-digestion method according to the prior art, surrounding tissue must be removed by digestion after the CT-scan, in order to achieve a 3D-model of the vascular system, the non-destructive method according to the invention allows, on the one hand, high-resolution pictures of the vascular system, and on the other hand, probes already scanned can subsequently still be used as the basis for histologic or electron-microscopic examinations, as surrounding tissue remains intact. Furthermore, the organ parts which are of great interest can be cut out and scanned at an even higher resolution. The image analysis then is carried out by the use of a suitable quantification software.

The advantageous use of the contrast agent has been proven for example for the morphometry of the kidney vasculature, including a quantification of kidney-glomeruli in mice, at a resolution of up to below 2.5 micrometers (voxel side size) (Shokiche, C. C. et al., 2016), as well as in the correlative imaging of the vascular system and muscle tissue of the hind extremities of the mouse (Schaad et al., 2017). In case of a perfusion of the animal body with Microfil® (Fluitec), so far the standard imaging method, normally a resolution of up to about 50-100 micrometers can be reached. In order to improve this resolution, the Microfil® was individually diluted by angiography technicians, which allowed a resolution of up to about 12 micrometers, depending on the degree of dilution. The perfusion of smaller vessels and capillaries, however, is insufficient, due to several factors, such as the higher viscosity (Perrien D. S: 2016).

The contrast agent according to the invention with the contrast solution, however, enters till into the smallest capillaries of up to 3-4 micrometers diameter, and thus allows a more detailed representation of the vascular system. The method of application according to the invention also offers a reproducible, low viscosity (compared to the various individual modifications of the Microfil® method).

A further advantage of the contrast agent according to the invention is that the polymerization results in an additional stability of the object to be tested, which is of advantage especially during the micro-CT-scan, as it improves the quality of imaging. Furthermore, the new contrast agent based on the contrast solution, has a high degree of absorption of x-rays, which is close to that of bone tissue. This facilitates a segmentation of the vessels to be represented based on thresholds, and their visualization.

The properties of casting- and auto-fluorescence of the contrast agent according to the invention therefore, in summary, allow a correlative approach, i.e. after the micro-CT-imaging and definition of the tissue sections to be further examined, in addition a morphologic analysis by histology and transmission-electron-microscopy can be carried out on the same test objects. The contrast agent according to the invention remains in the perfused blood vessels and is auto-fluorescent, which facilitates the “localization” of a specific histological section within the virtual stack of micro-CT-sections.

With respect to kidney-morphometry, neither the up-to-date upfield-MRI-techniques, nor the recently described method with “lightsheet microscopy” by means of in vivo anti-CD31-marking can offer a representation of the vascular tree of the kidney to the extent of the morphometric method based on micro-CT with the contrast agent according to the invention. Furthermore, this method allows a 3D-skeletonization and a corresponding analysis of the vascular system of organs by means of already publically available software. Besides the fast 3D-representation of vessels, the morphometry method based on micro-CT allows great savings of time (less than 24 hours versus 1-2 weeks in the classic method based on histology or the casting method).

Surprisingly, it has been found that the contrast agent according to the invention can also be used for the visualization of bone vessels after the decalcification. For the preceding decalcification, which is part of the prior art and not an object of the present invention, generally three main types of decalcifying agents can be used: firstly, those based on strong mineral acids, such as hydrochloric acid or nitric acid, secondly, those based on weaker, organic acids, such as formic acid (e.g. in a simple 10% aqueous solution or combined with formalin or with a buffer) or trichloroacetic acid, and thirdly, those which are composed of so-called chelating agents, e.g. a 10% EDTA solution. For the purpose of the visualization of bone vessels, preferably chelating agents are used. EDTA (ethylenediaminetetraacetic acid) has a slow effect, however, results in little tissue damage and standard coloring agents are hardly influenced. A possible composition of a decalcifying agent based on EDTA is a mixture of 250 g EDTA disodium salt and 1750 ml of distilled water, wherein the solution is adjusted to pH 7, preferably by the addition of about 25 g of sodium hydroxide.

Further embodiments of the invention are laid down in the dependent claims.

### BRIEF DESCRIPTION OF THE DRAWINGS

The contrast agent according to the invention has so far been used for example in the representation of the vasculature of the hind extremities of a mouse (see FIG. 1), as well as the vasculature of the kidney and glomeruli (FIG. 2-5).

Preferred embodiments of the invention, or examples for applications of the invention, respectively, are described in the following with reference to the drawings, which are for the purpose of illustrating the present preferred embodiments of the invention and not for the purpose of limiting the same. In the drawings,

FIG. 1 shows the vasculature of the lower hind extremity of a mouse, visualized by micro-CT; wherein A) shows a lateral 3D view, at a voxel-side length of 2.7 μm; B) a virtual transversal section of the vasculature (at the level shown in A), at a voxel-side length of 0.8, or 0.66 μm, respectively: tibia (T) and fibula (F) appear slightly colored due to their high x-ray absorption; in C-F, virtual transversal sections of the isolated soleus muscle (C, D) or of the plantaris muscle (E, F), respectively, are shown; in C′)-F′), specific sections are shown in more detail, marked by rectangles in C)-F); wherein the micro-vasculature is depicted in volume-rendering at a higher degree of magnification, with differing vascular density, tortuosity, and 3D-arrangement.

FIG. 2 shows different modalities of visualization of the kidney vasculature and the glomeruli; wherein in A), the reconstructed 3D-stack of the micro-CT data set is illustrated with focus on the kidney vasculature; in B, a virtual section through the data set is shown, by use of a different transfer function: the visualization is focussed on the kidney tissue; C-C′ show the visualization with focus on the glomeruli; figure C shows a volume-rendering of a virtual 500 μm thick disc, as shown by the white box at the bottom left; the white frame in C shows the point with the glomeruli in a larger magnification in figure C′; figure D-D′ shows the advanced visualization option: microangio-CT at a higher resolution (voxel side length=0.59 μm). The insert in figure D shows the virtual section level shown in D; the 3D-volume rendering of the micro vasculature of a glomerulus marked in D is shown in D′.

FIG. 3 correlative microscopy: Visualization of corresponding points by use of the micro-CT-data and the histological approach. After the imaging, the fixated kidney was further processed for the purpose of a histological section and examination; figures a-c show the visualisation of the same level (section) of the same kidney, by use of bright field- (a & a′) and fluorescence- (b & b′) microscopy, as well as micro-CT (c & c′). The green signal in b and b′ results from the autofluorescent contrast agent, which polymerizes in the vessels. This property facilitates the registration between histology and micro-CT based on the orientation on larger vessels. In figures a′-c′, the regions marked in a-c are shown at a higher magnification. The glomeruli are shown with circles in a′-c′.

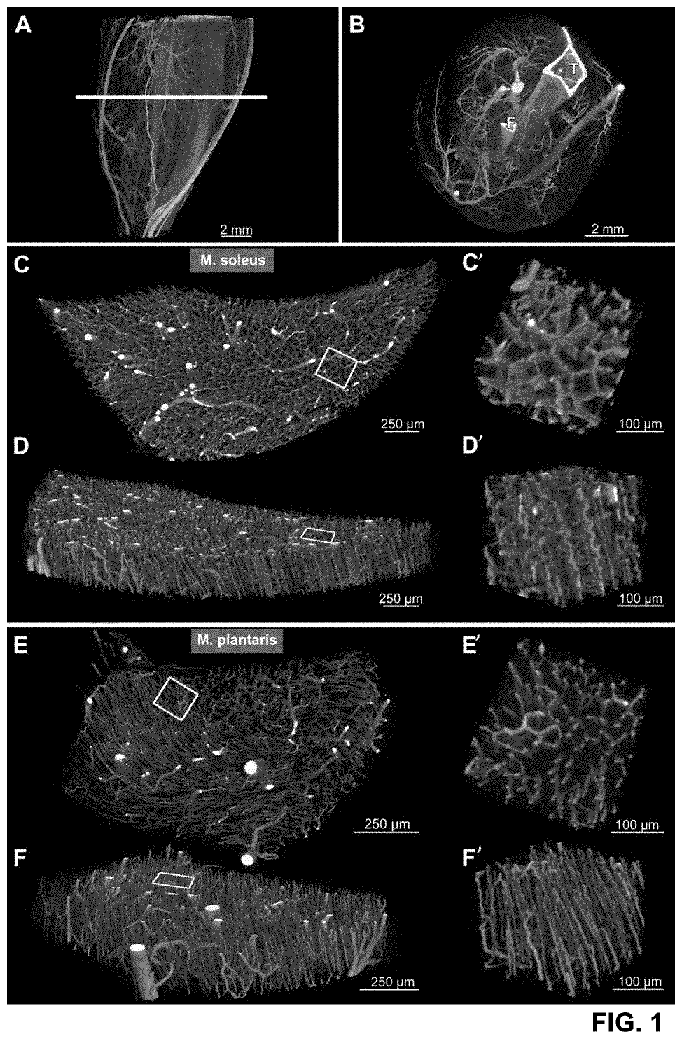

FIG. 4 Scheme of the combined fractionator/dissector principle used for the stereologic estimate of the number of glomeruli, based on (a) histological and (b) micro-CT data. a) classical histological principle: extensive sectioning of the entire kidney, in section pairs at 15 μm distance (dissector height in about 10 equidistant section sampling levels (SSL), about 1 mm). section thickness 5 μm. For the dissector height every third cut is used, and for the SSL, 197 sections (200-3) are discarded, until the next relevant dissector pair is used. On the corresponding images, which form a dissector, the glomeruli are counted, which appear in one section and not in the other, and which lie within the counting frame without touching the left and lower line (black checkmarks). The estimated number is multiplied by the inverse section sample factor (SSF) and area sample factors (ASF), in order to result in the absolute number of glomeruli per kidney based on the histology (Nabs-histo(glom), see text).
b) principle according to μaCT (microangio-CT): almost the same principle based on virtual picture stacks, of micor-CT-data. Dissector height (16 μm or 8 voxel height) and section pattern level (1 mm or 500 voxel height) are realized, in that the corresponding section numbers of the voxel stack are used (left side of the figure). Section/voxel height: 2 μm. All glomeruli on a total section level were counted according to the dissector-principle (check marks), such that no counting frame is necessary. The estimated number of glomeruli was only multiplied by the inversed section pattern factor, resulting in no necessity for the area-pattern ratio, in order to result in the total number of glomeruli per kidney (Nabs-μaCT(glom)).

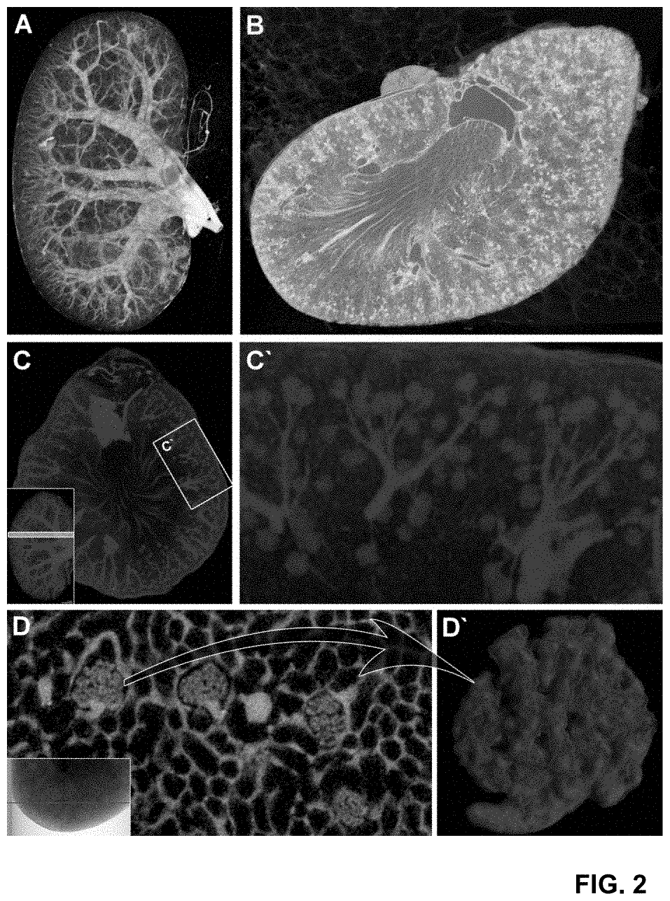

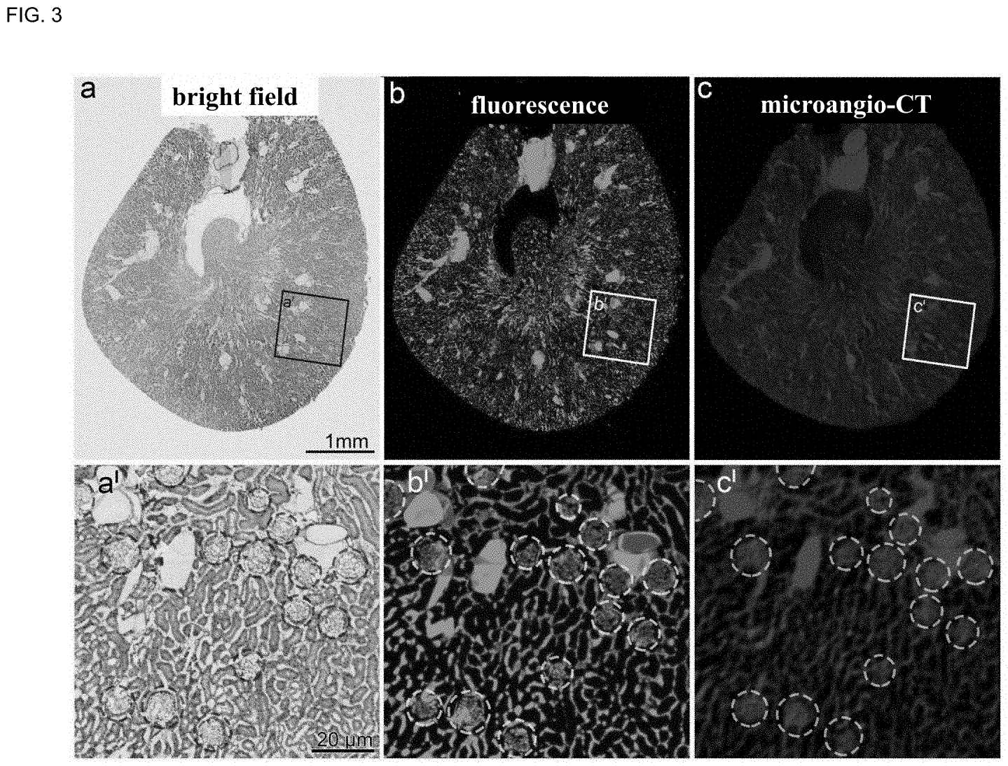

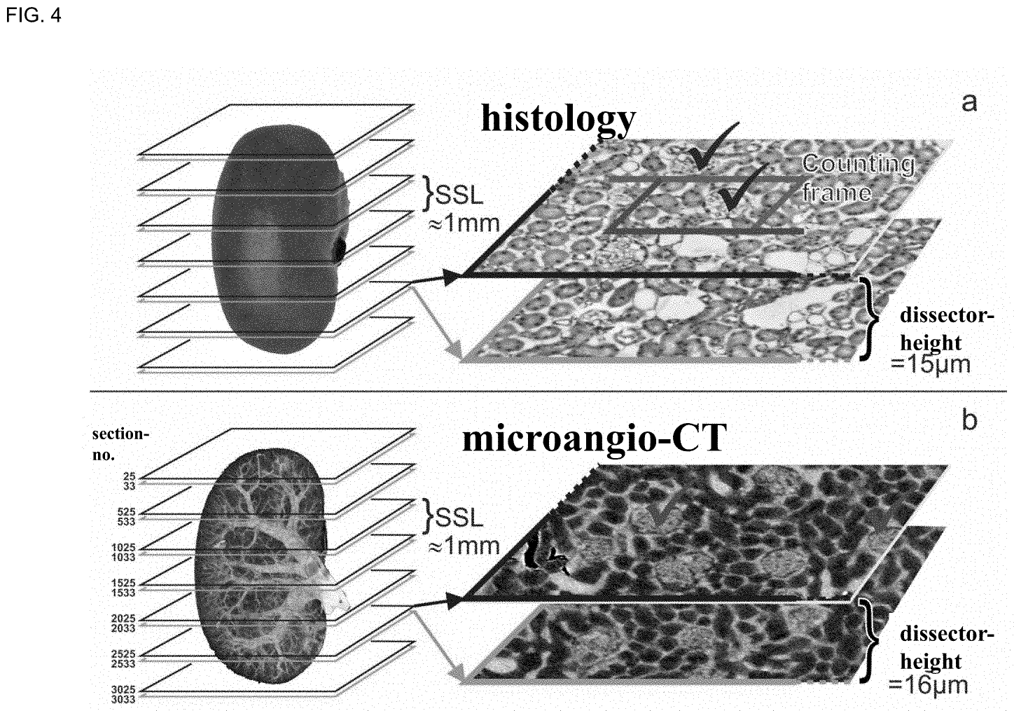

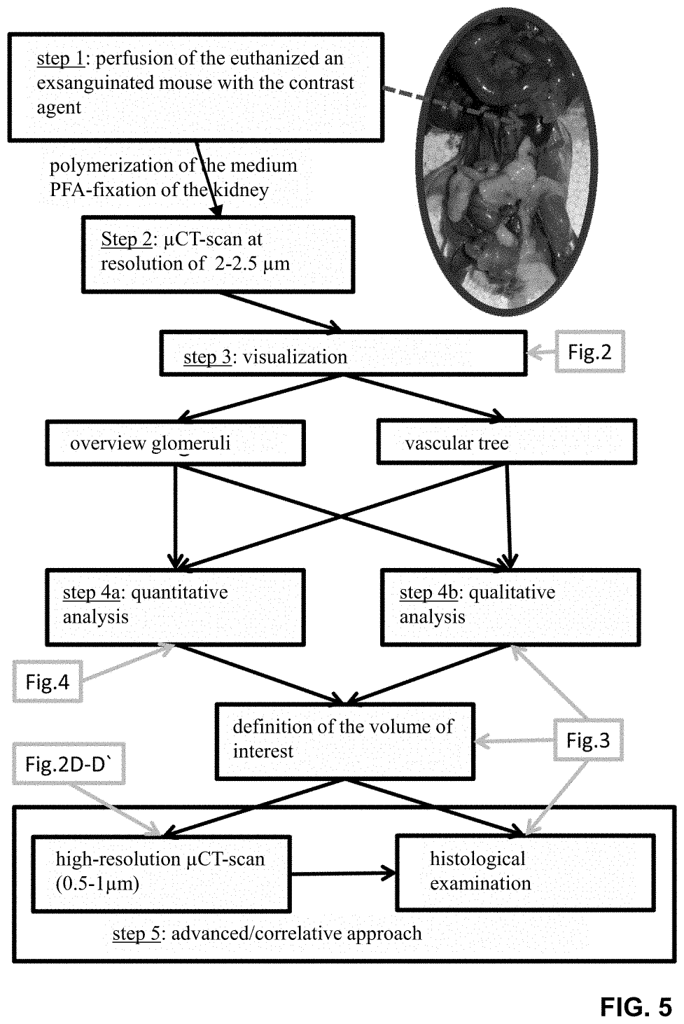

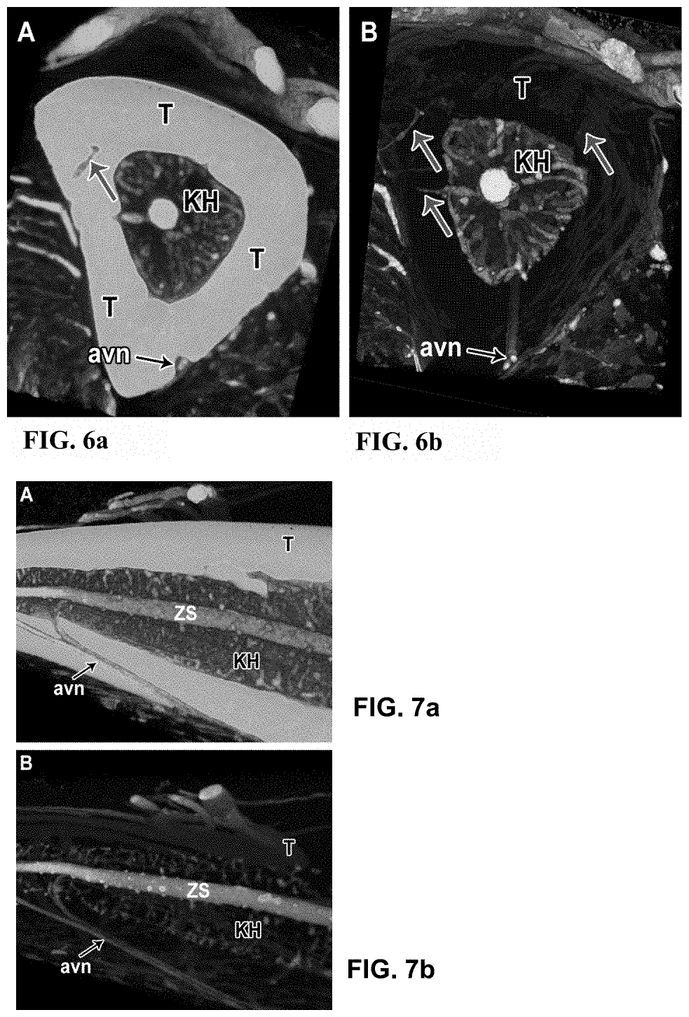

FIG. 5 Scheme of the suggested microangio-CT-method of the kidney (according to FIG. 2-4) by the use of the contrast agent according to the invention.

FIG. 6 Vasculature of the tibia bone of a mouse, illustrated by means of micro-CT by the use of the contrast agent according to the invention; (6a) and (6b) both show a virtual cross section through the same tibia bone, wherein in (6a) the bone is shown before and in (6b) after the decalcification with EDTA 10%. Due to the higher x-ray absorption, the tibia bone (T) appears lighter in (6a), and appears transparent in (6b) due to the lower x-ray absorption after the decalcification. Thus, the small connecting vessels running transversely through the bone tissue between periostal vessels and vessels of the medullar cavity (KH) are better visible and discoverable in (6b) (upwards directed arrows). The visualization of the vessels within the medullar cavity is visibly improved (6b) due to the lower x-ray absorption of the decalcified bone tissue of the tibia. On the outer edge of the tibia, the supplying arteries (avn=arteria and vena nutricia) are well visible.

FIG. 7 Vasculature of the tibia bone of a mouse, represented by micro-CT by use of the contrast agent according to the invention; (7a) and (7b) both show the virtual longitudinal section through the same tibia bone, wherein in (7a) the bone is shown before and in (7b) after the decalcification with EDTA 10%. Due to the higher x-ray absorption, the tibia bone (T) appears lighter in (7a), and appears transparent in (7b) due to the lower x-ray absorption after the decalcification. Thus, the bone vessels running through the bone tissue (avn=arteria and vena nutricia) are easier to follow in (7b), even though they are also visible in (7a). In the middle of the medullar cavity (KH), the central sinus (ZS) is well visible with its connections.

### DESCRIPTION OF PREFERRED EMBODIMENTS

1. Preparation of the single components:
   3 containers are provided. Container 1 contains a first mixture of iodized, esterified oil, preferably iodized, esterified linseed oil and 2-butanone (C₄H₈O), and a dye (BlueDye of VasQtec). This first mixture, whether with or without dye, is termed “contrast solution” within the context of this application. Container 2 contains the polyurethane (PU). Container 3 contains the hardener.
2. Removal of the contrast agent from container 1:
   Screwing-on of a 12 ml-disposable syringe (e.g. Monoject syringe with luer lock c1086, Qosina) onto Luer Connector (Needlefree Swabably Valve Female Luer to 20 mm Vial Cap Polycarbonate, Value Plastic) of container 1; injection of 5 ml of air into the container 1 (upright pressure), turning around of container 1, aspiration of the complete contrast agent into the syringe.
3. Production of a second mixture of contrast agent and polyurethane: injection of the contrast agent into the container 2 (which contains the PU); removal of the syringe and closure of the Luer Connector with Luer Cap.
4. Mixing of the second mixture:
   Mixing the second mixture in container 2 on a Vortex-device. The viscosity of the second mixture of contrast agent and PU (without hardener) which was used for the experiments so far is about 100 mPas·s at 20° C.
5. Aspiration of the second mixture in a syringe:
   Removal of the Luer Cap; injection of 10 ml of air into container 2; turning around of container 2 and aspiration of the second mixture into a 12 ml-disposable syringe.
6. Aspiration of the hardener:
   Screwing-on of a 1 ml-disposable syringe (1 ml syringe with luer slip c3302, Qosina) onto container 3 (or onto a Luer Connector fastened on container 3); injection of 0.5 ml of air into the container 3; aspiration of the entire hardener (1 ml).
7. Storage/removal of micro bubbles:
   For the de-airing of the contents of the syringes, i.e. for the removal of micro-bubbles, the syringes are brought into an upright position for at least 15 min.
8. Preparation of the mouse/rat:
   First, the animal is deeply anaesthetized or euthanized and then a cannula is inserted. The flushing of the animal body is carried out with 2×5-50 ml of clear solution, preferably with an isotonic solution, such as for example PBS (phosphate buffered saline) solution. Preferably, the animal is flushed in two halves (and then each half is also individually filled with contrast agent).
8a.) For the purpose of inserting the cannula and flushing of the lower half of the body, a cannula (e.g. BD Neoflon 0.6×19 mm, 26G of Aichele Medica AG) is inserted into the aorta, followed by subsequent antegrade perfusion of the clear solution; i.e. directed away from the heart. The puncture point into the aorta is preferably located in the thoracic region of the aorta. An indication for the filling of the lower half of the body is the inflation of the heart: if the heart begins to inflate, an incision is made in the right atrium. There, the clear solution then leaks out, first mixed with the flushed blood of the animal. As soon as the clear solution leaking from the atrium-incision is clear, one can assume that the lower half of the body is completely flushed.
8b.) For the purpose of inserting the cannula and flushing of the upper half of the body, a cannula is inserted in the same incision point, possible the same cannula as in step 8a in the reverse direction, wherein the perfusion is carried out in the same vessel, i.e. in the aorta, with the clear solution in a retrograde manner, i.e. towards the heart.
9. Fastening of the syringe:
   9a.) on the (manual) dispenser: insertion of the 12 ml syringe and the 1 ml syringe, each with its first end, in a 2-syringe-dispenser (two disposable syringes 11:1 (M-System, Medmix Systems AG) connected to each other; fastening of an adapter (Adapter L-System, Medmix Systems AG) in each case onto the second end of the two syringes; fastening of the adapter onto a mixing container (mixer, ML 3.2-16-LLM, DN3.2×16, Med. LuerLock, Medmix Systems AG); or
   9b.) on the injection pump: fastening of both syringes on the injection pump; manual adjustment of the correct position of the pump; unscrewing of the adapter in each case onto the second end of the two syringes; fastening of the adapter on the mixing container; set-up of the pump (Syringe Pump LEGATO 100, 220V/50 Hz, CE, kdScientific), selection of the parameters (Mode: infuse only; syringe: Sherwood 12 ml; flow rates: max. 1.5 ml/min; max. volume: about 3 ml/mouse, about 9-10 ml/rat).
10. Injection/perfusion:
   10a.) carefully and uniformly, with as uniform flow as possible, or
   10b.) start-up of pump, flow rate max. 1.5 ml/min

Filling of the Entire Lower and/or Upper Half of the Animal Body:

Now a cannula (e.g. BD Neoflon 0.6×19 mm, 26G, of Aichele Medica AG, optionally the cannula used for flushing) is fastened on the manual dispenser (option a) or on the pump (option b).

For the perfusion of the lower half of the body, as in the flushing step, the contrast agent is injected by an antegrade perfusion from the same injection point, which served as the flushing or exsanguination of the animal, respectively, into the animal body in the direction away from the heart. The discoloring of the lower extremities in the color of the contrast agent (preferably blue dye) serves as an indication for the filling of the lower half of the body.

For the filling of the entire upper half of the body with the contrast agent, as in the cannulation or flushing step, respectively, as described above, the contrast agent is now injected at the same injection point into the upper half of the animal body, i.e. by a retrograde perfusion of the contrast agent.

As a safeguard that no contrast agent flows into the heart, i.e. in order to prevent a dilatation of the left ventricle, the Aorta ascendens is bound closed with a thread. When the upper extremities, i.e. the paws, as well as the nose of the animal take on the color of the contrast agent, it can be assumed that the upper half of the animal body is completely filled.

For the filling of an entire body of a mouse or a rat, respectively, up to about 10 ml or up to about 35 ml of contrast agent are necessary.

Selective Filling of One or More Organs of the Animal Body:

All organs which belong to the lower half of the body, can be filled with contrast agent as described above by the complete filling of the lower half of the body. The brain can be filled with contrast agent by the entire filling of the upper half of the body.

The filling of the heart and/or of the lung, however, must be carried out selectively: for this purpose, a cannula is inserted into the aorta descendens, wherein distally of the aorta descendens, it is pinched off, such that only the aorta ascendens and the heart coronary vessels are filled. Then a clear solution (e.g. PBS) is injected only into this pinched-off part.

The vascular system of the lung is filled in a retrograde manner through the veins of the lung, which enter into the left atrium of the heart.

For the filling of selective organs, e.g. of the heart and/or of the lung, in comparison to the entire filling of the animal body, only about 0.5-1.5 ml of contrast agent are necessary.
11. Hardening:
   After the injection, the contrast agent dislocates from the aorta through the arteries into the capillary network and the venous system of the animal body and hardens there due to polymerization. The contrast agent should harden for at least 20-30 min. Subsequently, the animal body part with the hardened contrast agent is chemically fixated, preferably with 2% paraformaldehyde-solution, and can then be stored at 0-8° C. up to several months.
12. Image analysis by micro-CT-scan:
   The imaging method of the animal body, or the scanning by means of a micro-CT-device, is carried out in the hardened state. During the scanning, the body must not move/be moved, as this can result in disturbances in the pictures. In order to prevent smallest movements, the animal body shall be mechanically fixated during the scan.
   The test objects are scanned for the pictures shown in the figures by means of a “desktop micro-CT” device (SkyScan 1172 or 1272, Bruker, MicroCT, Kontich, Belgium).
13. Storage:
   After the CT-scan, the animal body can be stored again in a 2%-paraformaldehyde-solution at 0-8° C.
14. Histology:
   Subsequently, histological examinations of parts or organs of the animal body can be carried out. For this purpose, the conventional paraffin-embedding, the conventional histological section technique, dyes as well as immunohistochemistry can be used.
   Therein, the hardened contrast agent remains intravascular and is well visible, also after the histological sections. The autofluorescence of the contrast agent allows a direct comparison of the histological sections with the corresponding virtual sections of the micro-CT-data set.
15. Evaluation (quantitative and qualitative analysis of the micro-CT-data set):
   Micro-CT-projections can be projection-reconstructed backwards after scanning, e.g. by use of the NRecon software (NReconServer64 bit, Bruker, MicroCT, Kontich, Belgium), “volume-rendered” and three-dimensionally visualized by means of the CTVox software (Bruker, MicroCT, Kontich, Belgium). Tissue and blood vessels can be segmented and analyzed by use of the CTAn software (Bruker, MicroCT, Kontich, Belgium), or also determined by use of other publically available software, such as e.g. Matlab (The MathWorks, Inc., Natick, Mass., USA) or ImageJ (Rasband, W. S., ImageJ, U.S. National Institutes of Health, Bethesda, Md., USA, http://imagej.nih.gov/ij/, 1997-2016).

Mixing Tests:

In search for the optimal composition of the contrast agent different ratios of the iodized, esterified linseed oil to 2-butanone and to PU were tested. Therein, the preferred constant ratio of PU to hardener of 100:16 weight percent was used. In addition, in each case, 0.2 g (or a pinch, respectively), of a blue dye were used in powder form. The mixing tests were carried out as follows:

Each component was provided separately. The polyurethane (PU) was mixed in a glass container with the butanone, the iodized, esterified linseed oil, and the dye, by use of an ultrasound bath. Subsequently, the hardener was added and also mixed in the ultrasound bath for about 30 sec. The glass container with the mixture was then positioned in a vacuum chamber and one waited until small bubbles developed on the surface (about 40 sec.). Subsequently, a syringe was filled with the mixture and the mixture was injected into the object to be examined (perfusion).

For the determination of the viscosity, the mixture was submitted to a “drop fall test”. Each minute, 0.1 ml of the mixture was applied to a sheet of paper, which was held in a vertical position. The mixture passed through the paper. In order to measure the viscosity, the distance, which was passed by the mixture at specific points in time, was observed. A Venflon venous catheter was mounted on the syringe, in order to imitate the perfusion on the body. After the viscosity test, the venous catheter with the polymerized mixture was removed from the syringe. All venous catheters were examined in a micro-CT-device in terms of the absorption of the different mixtures.

| | | | | | iod., | | | |
|---|---|---|---|---|---|---|---|---|
| sample | | | 2- | | esterified | | | |
| no. | PU | hardener | butanone | | linseed oil | | dye | comment |
| 1a | 5 g | 0.8 ml | 2 | ml | 2.2 | ml | 1 pinch | minimum |
| 1b | 5 g | 0.8 ml | 2.2 | ml | 2.5 | ml | 1 pinch | |
| 1c | 5 g | 0.8 ml | 2.2 | ml | 3.0 | ml | 1 pinch | |
| 2 | 5 g | 0.8 ml | 2 | ml | 3.0 | ml | 1 pinch | |
| 3 | 5 g | 0.8 ml | 1 | ml | 3.0 | ml | 1 pinch | no perfusion |
| 4 | 5 g | 0.8 ml | 1.5 | ml | 3 | ml | 1 pinch | no perfusion |
| 5 | 5 g | 0.8 ml | 1.5 | ml | 3.5 | ml | 1 pinch | no perfusion |
| 6 | 5 g | 0.8 ml | 2 | ml | 3.5 | ml | 1 pinch | precipitation |
| 7 | 5 g | 0.8 ml | 2 | ml | 5 | ml | 1 pinch | precipitation |
| 8 | 5 g | 0.8 ml | 2 | ml | 7 | ml | 1 pinch | precipitation |
| 9 | 5 g | 0.8 ml | 2 | ml | 8 | ml | 1 pinch | Precipitation; |
| | | | | | | | | max. oil |

With respect to the polymerization, all except for three of the tested compositions were suitable for the perfusion. 10 minutes were determined as the minimal time for the perfusion. Samples 1a-c, 2, 6-9 fulfilled this requirement.

The tests showed that at least 2 ml of 2-butanone (for 5 g PU and 0.8 ml hardener) should be used. Below this value, the polymerization occurs too fast and thus does not allow a complete perfusion.

The amount of the iodized, esterified oil also influences the time of polymerization. Samples 8 and 9 showed a faster polymerization than the other samples. Thus, it seems as if with a constant volume of the PU, the hardener and the 2-butanone, 8 ml of the oil correspond to the maximal suitable concentration.

Samples 6-9 showed a precipitation of oil and dye after polymerization was completed, which could result in the diffusion of the contrast agent into the surrounding tissue, and possibly to the diminishing of the image quality.

Samples 8 and 9 showed a high concentration of the iodized oil and thus also a high absorption (possibly similar to bone tissue). This requires, for the reduction of the artifacts, the use of an aluminum filter for the scan, which leads to a longer scan time. A high absorption could however also influence the capillary recognition in a positive manner, however, it could also result in a supersaturation of the capillary-pixels, which would diminish the partial volume effect and possibly would allow a larger pixel size (in the experiments, in each case an isotropic pixel size of 0.8 μm was used).

The use of iodized, esterified poppy seed oil instead of iodized, esterified linseed oil showed a good perfusion, but a worse contrast in angiography, which probably is the result of the lower ratio of iodine. The use of acetone instead of butanone as a solvent showed similar effects as butanone, which is also expected from other ketones, such as for example diethylketone. A use of methylene chloride as an alternative solvent is also conceivable. The following measurements served the expression of the concentrations as percent by weight in the following table: 3.5 ml of iodized, esterified linseed oil weighed 4.8 g. 0.8 ml of hardener weighed 0.8 g. 2 ml of butanone weighed 1.5 g. The PU used had a density of about 1.05 g/cm³:

| | | | | iod., esterified |
|---|---|---|---|---|
| Sample | PU | hardener | butanone | linseed oil |
| 1a | 48.48% | 7.76% | 14.54% | 29.22% |
| 1b | 45.96% | 7.35% | 15.17% | 31.52% |
| 1c | 43.24% | 6.92% | 14.27% | 35.56% |
| 2 | 43.82% | 7.01% | 13.15% | 36.02% |
| 3 | 46.90% | 7.50% | 7.04% | 38.56% |
| 4 | 45.31% | 7.25% | 10.19% | 37.25% |
| 5 | 42.66% | 6.83% | 9.60% | 40.91% |
| 6 | 41.34% | 6.61% | 12.40% | 39.64% |
| 7 | 35.34% | 5.65% | 10.60% | 48.41% |
| 8 | 29.60% | 4.74% | 8.88% | 56.78% |
| 9 | 27.38% | 4.38% | 8.21% | 60.02% |

Optimization Tests:

After the mixing tests, the contrast agent mixture was further optimized. In the current, above described method for the injection of the contrast agent into the body to be examined, or the organ to be examined, respectively, the hardener, or the contents of the 3^{rd} container, respectively, is only admixed during or immediately prior to the injection of the remaining contrast agent components, respectively. For this purpose, a double syringe is used. This pre-determines a specific volume ratio of 1:11 between the hardener and the remaining (second) mixture. Thus, there is always an excess of hardener, which results in the fact that the amount of hardener in the total amount shall not be defining. In the mixing tests, no double syringe was used yet, which is why a defined amount of hardener of 0.8 ml was used.

The preferred range of volume ratio of PU to hardener is 100:16-100:19. However, this can also be varied and does not substantially influence the quality of the contrast agent. During the optimization tests, a volume ratio of the iodized, esterified linseed oil to 2-butanone of 54%/46% in the contrast agent was found to be optimal (under disregard of the optional dye due to its small amount) (options 2, 5, 6). However, the volume ratios of the iodized, esterified linseed oil to the 2-butanone of 53%/47% (option 1) or of 56%/44% (option 3) or even 58%/42% (option 4) show good results during the perfusion and subsequently show a good contrast in the imaging. Therefore, preferred ranges of the ratios of the volume of the iodized, esterified oil to the volume of the ketone can be defined, namely 0.75-4, preferably 1-1.5, especially 1.1-1.3.

The iodized, esterified oil influences the contrast. The butanone serves as a diluting agent. For a desired higher contrast, more iodized, esterified oil is added to the mixture, for less contrast, accordingly, less iodized, esterified oil. A relatively excessive amount of oil, results in a leakage of oil from the solution due to supersaturation and in a high viscosity, which hampers or prevents the perfusion, respectively.

As during the emptying of the individual containers, and during the mixing of the components and during the injection of the contrast agent, in each case, in the containers, as well as in the mixing tubes and the syringes, a residual volume remains on the walls or on the container floor, respectively, the volumes of the components in the optimization tests were optimized to the filling maximum of the containers (options 5, 6), at a constant optimal ratio of iodized, esterified linseed oil to 2-butanone (54%/46% of option 2). The actual filling amounts of the containers thus of course are to be adjusted to the corresponding container volume or according to the filling capacity depending on the object to be examined, respectively.

Options of the Contrast Agent Composition:

contrast agent-kit (option 1: minimum)

| | | | amounts of the | percent by |
|---|---|---|---|---|
| container | | amount | contrast solution | volume [%] |
| No. | | [ml] | components [ml] | (of total) |
| 1 | contrast solution: | 4.70 | 2.50 | 24.39 |
| | (iod. linseed oil, | | 2.20 | 21.46 |
| | 2-butanone, dye) | | 1 pinch | negligible |
| 2 | PU | 4.75 | | 46.34 |
| 3 | hardener | 0.80 | | 7.80 |
| total | | 10.25 | | 100 |

contrast agent kit (option 2: optimum)

| | | | amounts of the | percent by |
|---|---|---|---|---|
| container | | amount | contrast solution | volume [%] |
| No. | | [ml] | components [ml] | (of total) |
| 1 | contrast solution: | 4.80 | 2.60 | 25.12 |
| | (iod. linseed oil, | | 2.20 | 21.26 |
| | 2-butanone, dye) | | 1 pinch | negligible |
| 2 | PU | 4.75 | | 45.89 |
| 3 | hardener | 0.80 | | 7.73 |
| total | | 10.35 | | 100 |

contrast agent kit (option 3: maximum)

| | | | amounts of the | percent by |
|---|---|---|---|---|
| container | | amount | contrast solution | volume [%] |
| No. | | [ml] | components [ml] | (of total) |
| 1 | contrast solution: | 5.00 | 2.80 | 26.54 |
| | (iod. linseed oil, | | 2.20 | 20.85 |
| | 2-butanone, dye) | | 1 pinch | negligible |
| 2 | PU | 4.75 | | 45.02 |
| 3 | hardener | 0.80 | | 7.58 |
| total | | 10.55 | | 100 |

contrast agent kit (option 4, with adjusted
filling capacity, incl. volume loss)

| | | | amounts of the | percent by |
|---|---|---|---|---|
| container | | amount | contrast solution | volume [%] |
| No. | | [ml] | components [ml] | (of total) |
| 1 | contrast solution: | 6.3 | 3.65 | 27.65 |
| | (iod. linseed oil, | | 2.65 | 20.08 |
| | 2-butanone, dye) | | 1 pinch | negligible |
| 2 | PU | 5.80 | | 43.94 |
| 3 | hardener | 1.10 | | 8.33 |
| total | | 13.20 | | 100 |

contrast agent kit (option 5: optimum with adjusted filling capacity)

| | | | amounts of the | percent by |
|---|---|---|---|---|
| container | | amount | contrast solution | volume [%] |
| No. | | [ml] | components [ml] | (of total) |
| 1 | contrast solution: | 5.20 | 2.80 | 26.05 |
| | (iod. linseed oil, | | 2.40 | 22.33 |
| | 2-butanone, dye) | | 1 pinch | negligible |
| 2 | PU | 4.75 | | 44.19 |
| 3 | hardener | 0.80 | | 7.44 |
| total | | 10.75 | | 100 |

contrast agent kit (option 6: optimum with adjusted
filling capacity, incl. volume loss)

| | | | amounts of the | percent by |
|---|---|---|---|---|
| container | | amount | contrast solution | volume [%] |
| No. | | [ml] | components [ml] | (of total) |
| 1 | contrast solution: | 6.30 | 3.40 | 25.76 |
| | (iod. linseed oil, | | 2.90 | 21.97 |
| | 2-butanone, dye) | | 1 pinch | negligible |
| 2 | PU | 5.80 | | 43.94 |
| 3 | hardener | 1.10 | | 8.33 |
| total | | 13.20 | | 100 |

Viscosities:

| component | viscosity ([mPa s/20° C.] |
|---|---|
| iodized, esterified linseed oil | 85 |
| 2-butanone | 0.4 |
| PU | 6500 |
| hardener | 290 |

The viscosity of the second mixture, i.e. of the combination of contrast solution and PU (or of the contrast agent still without the hardener) is about 100 mPas·s at 20° C.

### REFERENCES

Zagorchev, L. et al., Micro computed tomography for vascular exploration, Journal of Angiogenesis Research 2010, 2:7
Meyer, E. et al., Polyurethane Elastomer: A New Material for the Visualization of Cadaveric Blood Vessels, Clinical Anatomy 20:000-000 (2007) (Wiley Interscience)
Krucker et al., New Polyurethane-Based Material for Vascular Corrosion Casting with Improved Physical and Imaging Characteristics. Res. Tech. 69: 138-147 (2006)
Meyer, E. et al., Altered morphology and 3D architecture of brain vasculature in a mouse model for Alzheimer's disease, PNAS, 105; 9; 3587-3592 (2008)
Grabherr et al., Angiofil®-mediated Visualization of the Vascular System by Microcomputed Tomography: A Feasibility Study, Microscopy Research and Technique 71(7): 551-556, 2008.
Shokiche, C. C., Baumann P., Hlushchuk R., Djonov V., Reyes M. (2016) High-Throughput Glomeruli Analysis of μ-CT Kidney Images Using Tree Priors and Scalable Sparse Computation. In: Ourselin S., Joskowicz L., Sabuncu M., Unal G., Wells W. (eds) Medical Image Computing and Computer-Assisted Intervention—MICCAI 2016.
Perrien, D. S. et al., Novel methods for microCT-based analyses of vasculature in the renal cortex reveal a loss of perfusable arterioles and glomeruli in eNOS−/− mice, BMC. Nephrol. 17, 24 (2016).
Schaad, L., et al., Correlative Imaging of the Murine Hind Limb Vasculature and Muscle Tissue by MicroCT and Light Microscopy, Scientific Reports, 7:41842 (2017), doi: 10.1038/srep4184

1. A contrast agent for ex vivo micro angiography, for the digital imaging of a vascular system of a mouse or a rat or other laboratory animals, or of individual animal- and human organs, by means of a micro-CT-device, comprising:
a polyurethane,
a hardener,
iodized, esterified oil, and
a ketone.

2. The contrast agent according to claim 1, wherein the iodized, esterified oil is an iodized, esterified poppy seed oil, or an iodized, esterified linseed oil.

3. The contrast agent according to claim 1, wherein the ketone is selected from 2-butanone, acetone, or 3-pentanone.

4. The contrast agent according to claim 1, wherein the contrast agent comprises a dye.

5. The contrast agent according to claim 1, wherein the polyurethane is an aliphatic isocyanate.

6. The contrast agent according to claim 1, wherein the hardener is a modified, aromatic diamine.

7. The contrast agent according to claim 1, wherein:
the iodized, esterified oil is contained at 20-60% in the contrast agent; and
the ketone is contained at 7-30% in the contrast agent.

8. The contrast agent according to claim 1, wherein in a contrast solution, which comprises the iodized, esterified oil and the ketone, the ratio of the volume of the iodized, esterified oil to the volume of the ketone is 0.75-4.

9. The contrast agent according to claim 1, wherein the polyurethane is contained at 25-60% in the contrast agent; and
wherein the hardener is contained at 4-10% in the contrast agent.

10. A kit-of-parts for ex vivo micro angiography, comprising:
a first container, comprising an iodized, esterified oil, and containing a ketone;
a second container, comprising a polyurethane; and
a third container, comprising a hardener.

11. The kit-of-parts according to claim 10, wherein the first container additionally comprises a dye.

12. The kit-of-parts according to claim 10,
wherein the first container comprises a first mixture of 2-4 ml of the iodized, esterified oil and 2-3 ml of the ketone; wherein the second container comprises 4-7 ml of the polyurethane; and
wherein the third container comprises 0.5-1.5 ml of the hardener.

13. The kit-of-parts according to claim 10, further comprising:
a first syringe, for receiving the contents of the first container and the second container;
a second syringe, for receiving the contents of the third container; and
a mixing container for mixing the contents of the first syringe and the second syringe.

14. A method for the production of a contrast agent for ex vivo micro angiography according to claim 1, for the digital imaging of a vascular system of a mouse or of a rat by means of a micro-CT-device, comprising the following steps:
providing a first mixture of iodized, esterified oil, with a ketone, in a first container;
providing a polyurethane in a second container;
providing a hardener in a third container;
blending and mixing of the contents of the first container with the contents of the second container to form a second mixture; and
blending of the contents of the third container with the second mixture in a mixing element, immediately prior to the injection into the vascular system to be examined.

15. The method for ex vivo micro angiography for the digital imaging of a vascular system of an animal or human body or organ, by means of a micro-CT-device, comprising the following steps:
providing a contrast agent according to claim 1;
inserting a cannula and flushing of the animal body to be examined; and
injecting the contrast agent into the body or into the organ, respectively.

16. The method for ex vivo micro angiography according to claim 15, wherein after the injection of the contrast agent, a hardening of the contrast agent in the animal body is waited out, and subsequently the animal body is scanned by means of a micro-CT-device.

17. The method for ex vivo micro angiography according to claim 15, wherein the injection in step m) is carried out manually, or that the injection in step m) is carried out by means of an injection pump.

18. The method for using a contrast agent according to claim 1 for the post-mortem micro angiography, wherein the contrast agent is injected into a human or animal body or into a human or animal organ.

19. The contrast agent according to claim 2, wherein the iodized, esterified linseed oil is ethyl-9,12,15-triiodo-octadecatrienoate.

20. The contrast agent according to claim 2, wherein the iodized, esterified linseed oil is ethyl-9,12,15-triiodo-octadecatrienoate.

21. The method for using a contrast agent of a kit-of-parts according to claim 10 for the post-mortem micro angiography, wherein the contrast agent is injected into a human or animal body or into a human or animal organ.
